(19) United States
(12) Patent Application Publication
(10) Pub. No.: US 2026/0262986 A1
(43) Pub. Date: Sep. 10, 2026

(54) INTAKE AND OUTPUT REGISTER

(71) Applicant: Alice Hallie MAYES, Salem, VA (US)

(72) Inventor: Alice Hallie MAYES, Salem, VA (US)

(21) Appl. No.: 19/559,200

(22) Filed: Mar. 6, 2026

Related U.S. Application Data
(60) Provisional application No. 63/768,928, filed on Mar. 8, 2025.

(51) Int. Cl. A61B 5/20

(52) U.S. Cl. CPC A61B 5/208

(57) ABSTRACT

A system for real-time documentation of patient fluid intake and output events, including: a first register device configured to record fluid intake events at a point of patient care; and a second register device configured to record fluid output events at a point of patient care, wherein each of the first and second register devices comprises one or more input controls configured to record a fluid event by incrementing a cumulative value by a predetermined unit amount, a display configured to present a cumulative total of recorded fluid events, and a correction control configured to adjust the cumulative total to account for an erroneously recorded fluid event, and wherein the first and second register devices are configured to allow recording of fluid intake and output events substantially contemporaneously with occurrence of the events without requiring entry into an electronic medical record system.

### CROSS-REFERENCE TO RELATED APPLICATION

This application claims priority from and the benefit of U.S. Provisional Patent Application No. 63/768,928, filed on March 8, 2025, which is hereby incorporated by reference for all purposes as if fully set forth herein.

### BACKGROUND

### Field

Exemplary embodiments relate to generally to patient care documentation devices, and more particularly to dedicated hardware systems for real-time recording of patient fluid intake and output events at the point of care.

### Discussion on the Background

Patient intake and output refer to the measurement and monitoring of the fluids that a medical patient takes in and excretes over a specific period. For example, doctors may need to know patient intake and output in order to adjust treatments for heart failure patients and prescribe the correct dose of medication. There may be a problem with getting accurate input and output numbers, so patients may have extended stays in the hospital and often return after being discharged due to insufficiently prescribed medications. Accordingly, problems with intake and output measurement and monitoring may contribute to prolonged inpatient stays, repeat hospitalizations, and increased cost of medications, and thus may create significant economic impacts.

It is reported that hospitals lose an average of $1000 per visit on heart failure patients (Osenenko, Katherine M., Kuti, Effie, et al., 2022), and just in the United States there are an estimated 1,270,360 hospitalizations each year (Clark, Katherine A.A., Reinhardt, Samuel W., et al., 2022). There may also be an impact on health outcomes for patients who require accurate monitoring of their intake and output. These patients may suffer from complications as a result of poor fluid management.

Tracking intake and output may be a significant burden on time and effort by healthcare providers. Physicians need accurate intake and output measurements to adjust medications. Inaccuracy in intake and output measurements may prolong patient hospital stays and increase likelihood patients will return to the hospital after failed treatment after discharge. For example, when a patient is admitted to the hospital for a diagnosis such as renal failure or heart failure, the intake and output is what helps the physician order the correct dose of diuretic medication that the patient will be able to be discharged on. When an output is not documented properly, then physicians must rely on daily weights to adjust the patient’s medication. This means physicians may have to wait a full twenty-four hours to get the data they need. Then with every medication adjustment they may have to wait an additional twenty-four hours to make any further changes or determine if the prescription is correct for discharge. This cycle can delay a patient’s discharge for days or even weeks. This may create a significant financial burden for the patient, hospital, government, or insurer.

This problem with intake and output measurement and monitoring is currently addressed by policies and procedures created to optimize workflow, communication, and adjust practices for staff shortages. Nursing staff typically measure and document the type and amount of intake and output in a patient’s chart. Healthcare facilities may develop a policy or procedure for intake and output measurement and monitoring. For example, intake and output must be documented every two hours. The staff member who disposes of urine or removes a patient's tray from a hospital room may be responsible for this documentation. Most of the time the primary nurse is ultimately responsible for ensuring the intake and output is documented.

There may be unrealistic expectations on healthcare staff to maintain accurate intake and output measurement and monitoring. Staff may be desensitized to the pressures placed on them by doctors and leadership. There may be staff shortages, time constraints, inadequate training, poor communication, complex documentation systems, lack of awareness regarding the importance of intake and output measurement and monitoring, patient factors like confusion or incontinence, and challenges in accurately measuring fluid intake from various sources. All of these can lead to inaccurate recording and missed opportunities to identify potential fluid imbalances.

For example, if a patient calls out to go to the restroom the staff person may assist the patient to the restroom. The process may involve sanitizing hands, putting on gloves, walking to the restroom with the patient, making sure something is in the toilet to catch the urine, assisting the patient to sit and stand up again, assisting the patient with cleaning themselves, assisting the patient back to bed, remembering to put the bed alarm on and other adjustments to the bed. Then the staff person may need to pour out the urine, take gloves off, sanitize hands, get a pen and immediately write output measurements down or try to remember measurements until they can log into a computer.

There are many things that may go wrong with the intake and output measurement and monitoring process potentially leading to lost data, including forgetting the measurement, losing measurement notes, or forgetting to chart notes. Another problem is nurse assistants may do a large portion of the tasks involved with intake and output measurement and monitoring but the primary nurse is the one ultimately responsible for recording measurements, potentially leading to staff miscommunication. Further, a hospital nurse can be interrupted anywhere from several times to up to twenty times per hour depending on the unit and workload, which may lead to potential errors in intake and output measurement and monitoring due to distractions and multitasking.

The above information disclosed in this Background section is only for enhancement of understanding of the background of the inventive concept, and, therefore, it may contain information that does not form the prior art that is already known in this country to a person of ordinary skill in the art.

### SUMMARY

Exemplary embodiments provide dedicated intake and output register devices configured for real-time documentation of patient fluid events at the point of care.

Exemplary embodiments also provide hardware-based systems including dedicated intake and output register devices for real-time recording of patient fluid intake and output events in clinical environments.

Exemplary embodiments also provide purpose-built register devices that enable contemporaneous recording of patient fluid intake and output events without reliance on active electronic charting sessions.

Additional aspects will be set forth in the detailed description which follows, and, in part, will be apparent from the disclosure, or may be learned by practice of the inventive concept.

According to exemplary embodiments, a system for real-time documentation of patient fluid intake and output events includes a first register device configured to record fluid intake events at a point of patient care and a second register device configured to record fluid output events at a point of patient care, wherein each of the first and second register devices includes one or more input controls configured to record a fluid event by incrementing a cumulative value by a predetermined unit amount, a display configured to present a cumulative total of recorded fluid events, and a correction control configured to adjust the cumulative total to account for an erroneously recorded fluid event, and wherein the first and second register devices are configured to allow recording of fluid intake and output events substantially contemporaneously with occurrence of the events without requiring entry into an electronic medical record system.

According to exemplary embodiments, an intake and output register system for tracking a patient’s fluid balance includes a first register configured to record fluid intake and a second register configured to record fluid output, wherein the first register includes a first display screen for displaying total intake and times of incontinence, a plurality of first input buttons corresponding to numerical values for recording intake amounts and incontinence events, a first clear/reset button for resetting the recorded intake to zero, a plurality of first correction buttons for modifying erroneously entered data, and a first lock-out button to prevent unintended input when activated, and wherein the second register includes a second display screen for displaying total output and times of incontinence, a plurality of second output buttons corresponding to numerical values for recording output amounts and incontinence events, a second clear/reset button for resetting the recorded output to zero, a plurality of second correction buttons for modifying erroneously entered data, and a second lock-out button to prevent unintended input when activated.

According to exemplary embodiments, a patient fluid documentation system includes a first register device configured to record fluid intake events at a point of patient care, a second register device configured to record fluid output events at a point of patient care, and a communication interface integrated into at least one of the first register device or the second register device and configured to transmit recorded intake and output data from the respective register device to a remote computing device, wherein each of the first and second register devices includes a housing, one or more input controls configured to increment a cumulative value by a predetermined unit amount, a display configured to present the cumulative value, and a correction control configured to adjust the cumulative value.

The foregoing general description and the following detailed description are exemplary and explanatory and are intended to provide further explanation of the claimed subject matter.

### BRIEF DESCRIPTION OF THE DRAWINGS

The accompanying drawings, which are included to provide a further understanding of the inventive concept, and are incorporated in and constitute a part of this specification, illustrate exemplary embodiments of the inventive concept, and, together with the description, serve to explain principles of the inventive concept.

FIG. 1 is a front view of an intake register of an intake and output register according to an exemplary embodiment.

FIG. 2 is a front view of an output register of the intake and output register according to the present exemplary embodiment.

FIG. 3A is a top view of the intake and output register according to the present exemplary embodiment.

FIG. 3B is a front view of a mount for the intake and output register according to the present exemplary embodiment.

FIG. 4 is a perspective view of a hospital room with the intake and output register according to the present exemplary embodiment.

FIG. 5 is a perspective view of a hospital room bathroom with the intake and output register according to the present exemplary embodiment.

FIG. 6 is a front view of an intake register of an intake and output register according to an exemplary embodiment.

FIG. 7 is a front view of an output register of the intake and output register according to the present exemplary embodiment.

### DETAILED DESCRIPTION OF THE ILLUSTRATED EMBODIMENTS

In the following description, for the purposes of explanation, numerous specific details are set forth in order to provide a thorough understanding of various exemplary embodiments. It is apparent, however, that various exemplary embodiments may be practiced without these specific details or with one or more equivalent arrangements.

The terminology used herein is for the purpose of describing particular embodiments and is not intended to be limiting. As used herein, the singular forms, "a," "an," and "the" are intended to include the plural forms as well, unless the context clearly indicates otherwise. Moreover, the terms “comprises,” comprising,” "includes," and/or "including," when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and will not be interpreted in an idealized or overly formal sense, unless expressly so defined herein.

The intake and output register according to the present exemplary embodiment removes data entry barriers and streamlines the process of documenting intake and output. The processes used in hospitals require people to measure and document amounts in electronic medical records (EMR), which as discussed above are complicated processes with many barriers to success.

Removing these barriers to intake and output documentation requires a solution where people can document in real time without stepping away from a patient. Like checking out at the grocery store, the intake and output register only requires the person performing the action to input the relevant data at the time of care. As long as that is done the data is safely recorded. Data is not lost because a staff person forgot to chart it, for example. The data will stay on the device until being manually transferred to an EMR or automatically populated into the patient’s chart via integrated EMR software. This simplifies the nurse’s task to clearing the intake and output register or acknowledging the populated data in the EMR.

In particular, the intake and output register allow a healthcare provider to quickly register a patient’s data and move on with their work, so less time may be required to complete intake and output documentation, and registering data using the intake and output register is simple and easy to learn. The intake and output register is readily available, so for example, if a staff person empties urine or picks up a cup that staff person can register it in the intake and output register at that time.

The intake and output register according to the present exemplary embodiment is readily available to a user and does not lose data until the data is cleared or transferred to a patient’s medical chart. Instead of documenting small amounts of data several times the user can clear and document data every two or four hours in the intake and output register, for example, depending on needs of the medical provider. The intake and output register may also increase awareness of medical providers. Providers may recognize the device from one facility to the next.

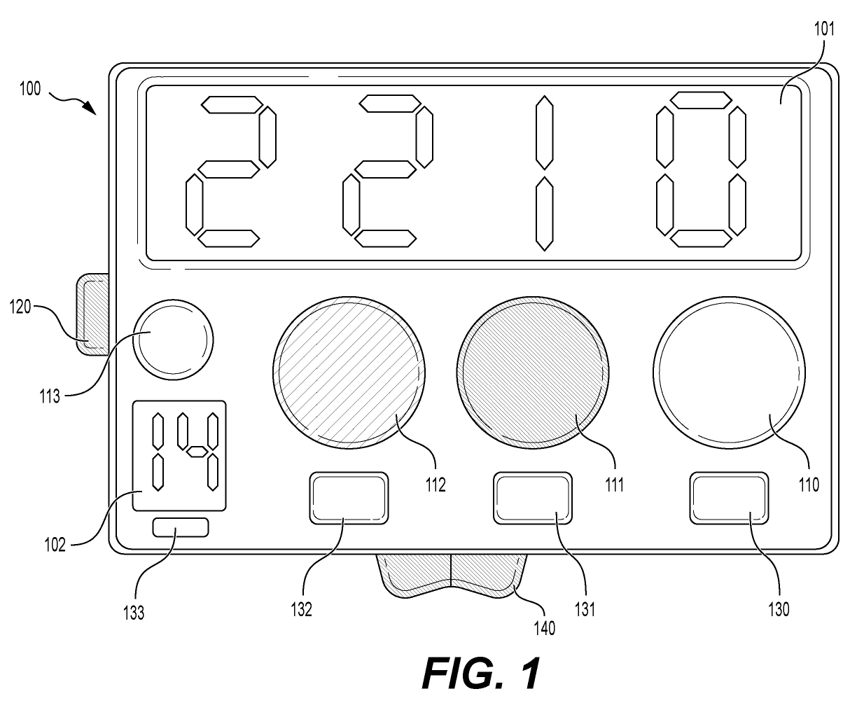

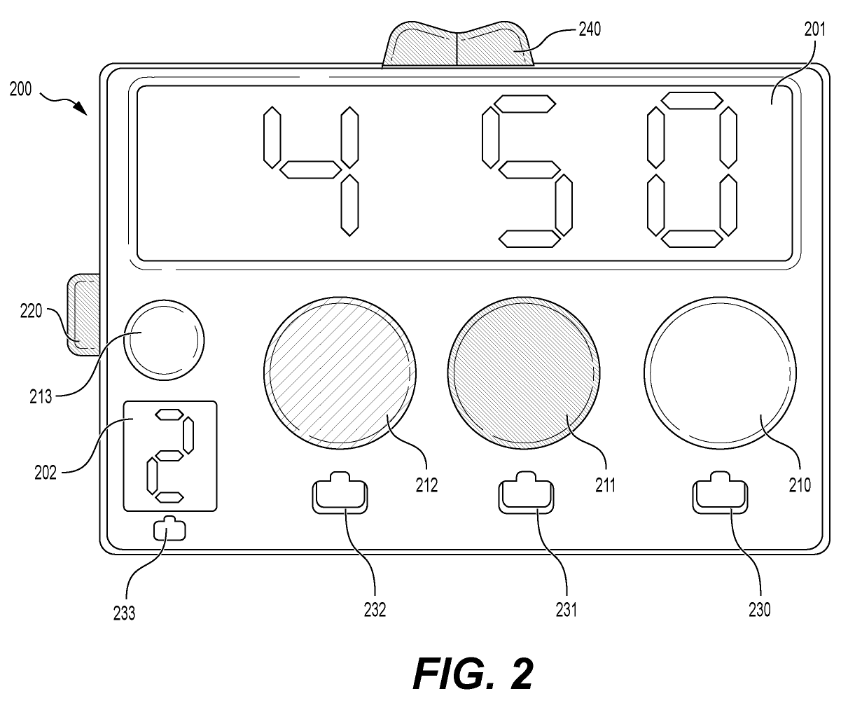

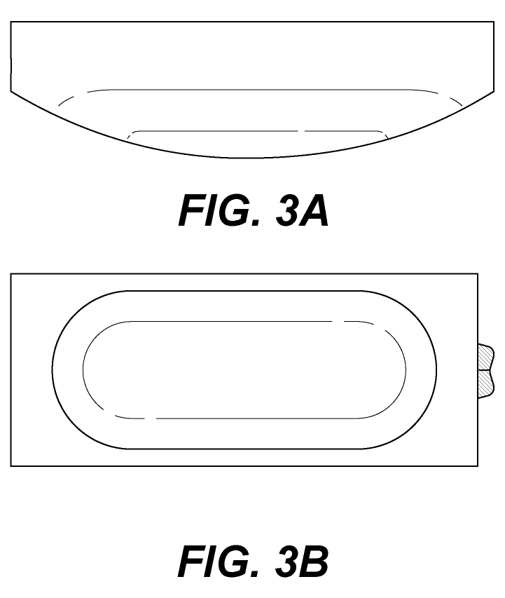

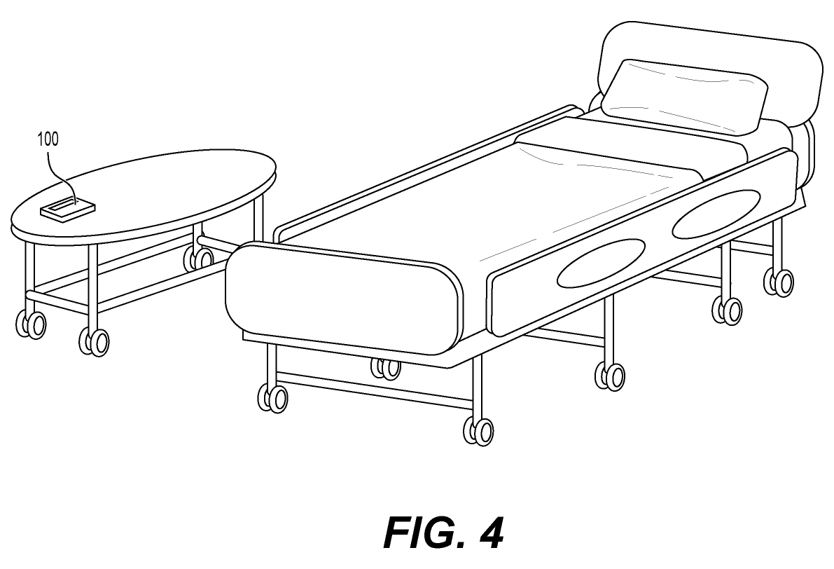

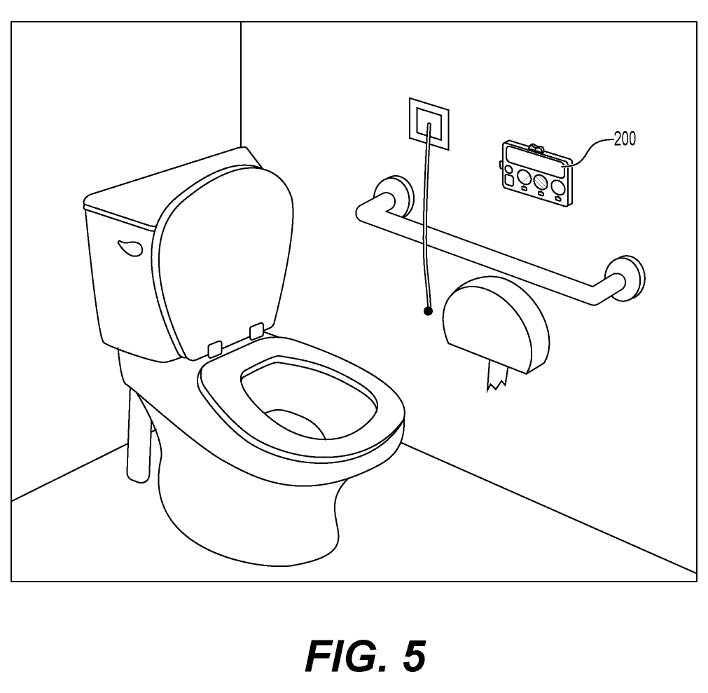

As shown in FIG. 1, FIG. 2, FIG. 3A, FIG. 3B, FIG. 4, and FIG. 5, the intake and output register according to the present exemplary embodiment is two separate devices, that is, an intake register 100 and an output register 200. Alternatively, the intake and output register may be a single device that tracks both a patient’s intake and output. The intake register 100 may be located at the patient’s bedside, such as on a bedside table where the patient has intake. The output register 200 may be attached to a wall in the bathroom where the patient’s output is disposed of. If the intake and output register is a single device, a user may transport it between intake and output locations, and the intake and output register may be secured in a separate cradle mount (described below) situated in each location.

The intake and output register according to the present exemplary embodiment may respectively be substantially equivalent in composition. The intake register 100 is described below, and for the sake of brevity repeated description of the output register 200 is omitted. The intake register 100 has a display screen 101, displaying total intake. The display screen 101 shows numbers being added when buttons (as described below) are activated by a user, then return to show total counts. The incontinence count tracks a patient’s times incontinent so the medical provider is aware and can adjust the patient’s care as appropriate. Alternatively, the intake and output register may have two display screens, one displaying total intake and one displaying times incontinent. According to the present exemplary embodiment, the input register 100 has another display screen 102 showing total episodes of unmeasured incontinence.

The input register 100 has four count buttons 110, 111, 112, and 113, one 113 for times incontinent and three for tracking intake. The intake buttons 110, 111, and 112 represent the ones, tens, and hundreds place, respectively. The input register 100 has a clear/ reset button 120 on the side thereof, for example where a right-handed user may place their thumb. The clear/ reset button 120 may be pushed to bring the count down to a zero status. The count may be zeroed when intake amount data is documented into the patient’s chart or manually or electronically transferred into the EMR.

Correction buttons 130, 131, 132, and 133 are located under each intake count button 110, 111, and 112 and incontinence count button 113, respectively, so that the user can correct the data he or she enters in the intake register 100, for example if accidentally entering data corresponding to too much patient intake. According to the present exemplary embodiment, the display screen 101 has a five-second delay timer before returning to the total count screen. If an erroneous data entry is not corrected before the delay timer expires, the user may need to reset the intake register to zero and manually enter the correct amount into the EMR.The lock-out button 140 is an optional button which can be activated if there is a risk for the patient to push buttons on the intake register 100, for example. When the lock-out button 140 is pressed all other buttons will not respond when pushed. The user will push the lock-out button 140 again to reactivate the other buttons.

The intake and output register according to the present exemplary embodiment has a curved magnetic back surface, as shown in FIG. 3A. The intake and output register has a cradle mount (i.e., a mounting bracket) that is curved, as shown in FIG. 3B, and is configured to seat the intake and output register, and has a metal plate embedded to secure the intake and output register’s magnetic surface. The cradle mount can be secured to a surface with adhesive and or screws.

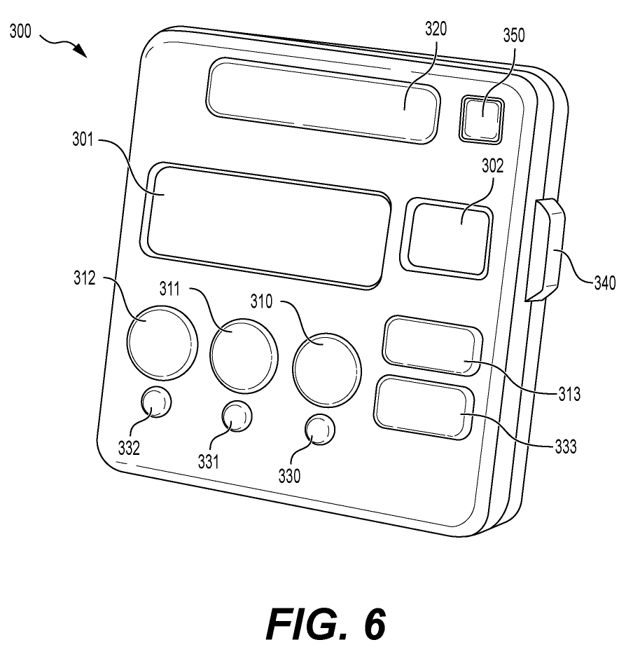

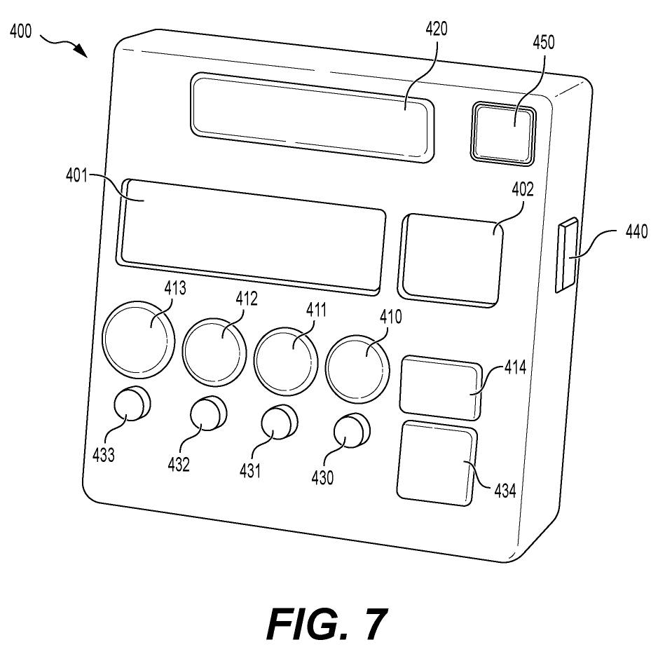

An intake and output register according to an exemplary embodiment is shown in FIG. 6 and FIG. 7. The intake and output register according to the present exemplary embodiment may be substantially similar in various respects to the intake and output register as described with respect to FIG. 1, FIG. 2, FIG. 3A, FIG. 3B, FIG. 4, and FIG. 5 of the present application, and the disclosure thereof is incorporated herein by reference, and any repeated disclosure may be omitted for the sake of brevity. Likewise, the disclosure with respect to the intake and output register described in connection with FIG. 6 and FIG. 7 is incorporated by reference into the exemplary embodiment described with respect to FIG. 1, FIG. 2, FIG. 3A, FIG. 3B, FIG. 4, and FIG. 5.

The intake and output register according to the present exemplary embodiment is two separate devices, that is, an intake register 300 and an output register 400. The intake register 300 is described below, and for the sake of brevity repeated description of the output register 400 is omitted. The intake register 300 has a display screen 301, displaying total intake, and another display screen 302 showing total episodes of unmeasured incontinence. The input register 300 has four count buttons 310, 311, 312, and 313, one 313 for times incontinent and three for tracking intake 310, 311, and 312. The input register 300 has a clear/ reset button 320 on a front face thereof. Correction buttons 330, 331, 332, and 333 are located under each intake count button 310, 311, 312, and incontinence count button 313, respectively. The intake register 300 may have a lock-out button 340.

As shown in FIG. 7, the output register 400 has five count buttons 410, 411, 412, 413, and 414, one 414 for times incontinent and four for tracking intake 410, 411, 412, 413. Correction buttons 430, 431, 432, 433, and 434 are located under each intake count button 410, 411, 412, and 413, and incontinence count button 415, respectively.

According to the present exemplary embodiment, as shown in FIG. 6 with respect to the input register 300, the intake and output register system may further include a dedicated input and output indicator 350, which may be a strict input and output indicator, configured to provide a clear, persistent visual signal that a patient is subject to strict intake and output documentation requirements. The input and output indicator 350 may be physically integrated into the housing of one or both of the intake and output register devices and may be positioned so as to be readily visible to caregivers in the vicinity of the patient’s bed or associated care area. The input and output indicator 350 may comprise a flush-mounted light element disposed on a front-facing portion of the register housing, adjacent to the display and input controls, so that the input and output indicator 350 is visible at a glance during routine patient care activities.

The input and output indicator 350 may be configured as a distinctively colored illumination source, such as a blue light, and may be provided in a square or other clearly recognizable shape, optionally accompanied by labeling identifying the intake and output status. The color, shape, and labeling are selected to distinguish the indicator from other visual signals commonly present in clinical environments, thereby reducing the likelihood of confusion and enabling caregivers to immediately recognize that enhanced documentation attention is required for the associated patient. The input and output indicator 350 may include diffused illumination or backlighting to enhance visibility under varying ambient lighting conditions.

The input and output indicator 350 may serve as a workflow-governing feature that reinforces the requirement for real-time documentation of fluid intake and output events. When activated, the input and output indicator 350 may provide a persistent reminder to caregivers that all intake and output events should be recorded promptly using the associated register devices, rather than deferred for later entry into a charting system. This visual cue is intended to reduce reliance on memory, minimize missed entries, and improve compliance with strict intake and output protocols.

Activation of the input and output indicator 350 may occur in several ways. According to an exemplary embodiment, the input and output indicator may be manually activated by a caregiver using a dedicated control on the register device or by setting the device into a strict monitoring mode. According to another exemplary embodiment, activation may be triggered automatically in response to an external signal, such as a status designation transmitted from an electronic medical record (EMR) system indicating that the patient has been placed on strict intake and output monitoring. The register device may include communication circuitry configured to receive status signals from a central system and to activate or deactivate the indicator accordingly.

The input and output indicator 350 may remain illuminated continuously while the strict monitoring status is active, and may be deactivated manually or automatically when the strict intake and output requirement is discontinued. According to an exemplary embodiment, the input and output indicator 350 may also be linked to other operational features of the register device, such as enabling or emphasizing certain display elements or prompting periodic review or reconciliation of recorded values.

By integrating the input and output indicator 350 directly into the intake and output register hardware, the system provides a physical, context-specific signal that is closely associated with the act of input and output documentation itself. This arrangement differs from conventional nurse-call or room-status indicator lights, which are typically intended to communicate patient requests or room conditions rather than to govern documentation workflow. The input and output indicator 350 thus functions not merely as a status light, but as an integral component of a system designed to improve the reliability and timeliness of fluid intake and output documentation at the point of care.

According to an exemplary embodiment, the intake and output register system may further include data transmission and connectivity capabilities configured to permit recorded intake and output information to be communicated from the register devices to external systems for storage, review, and integration with clinical documentation workflows. Each of the intake and output register devices may include an internal memory component for storing recorded values corresponding to fluid intake amounts, fluid output amounts, and unmeasured or incontinent events. The stored data may be retained within the device for later retrieval, or may be transmitted periodically or on demand to one or more external systems.

According to an exemplary embodiment, the register devices may include wireless communication circuitry configured to enable transmission of recorded data to a remote computing environment. Such communication circuitry may utilize one or more suitable wireless communication protocols, including but not limited to Wi-Fi, Bluetooth, cellular communication, or other short-range or long-range wireless technologies. The communication module may be integrated within the device housing and configured to operate automatically in the background without requiring active caregiver intervention, thereby minimizing disruption to clinical workflows.

The transmitted data may be communicated to a centralized server or cloud-based storage system configured to receive, store, and organize intake and output records associated with a particular patient. According to an exemplary embodiment, the system may associate transmitted data with patient identification information so that the recorded intake and output values can be retrieved and reviewed in connection with other patient records. The cloud-based system may further provide secure access to authorized users, such as nurses, physicians, or other healthcare providers, allowing remote review of intake and output data in near real time.

According to an exemplary embodiment, the register devices may be configured to communicate with an electronic medical record (EMR) system. In such implementations, recorded intake and output values may be transmitted from the register devices to the EMR system in a preliminary or pending state rather than being immediately entered as final chart entries. This pending-state transmission enables caregivers to review, confirm, and reconcile the recorded values within the EMR workflow prior to final documentation. Such a configuration may align the register system with established clinical documentation practices in which entries are verified before being committed to the permanent patient record.

The system may further support periodic synchronization of data between the register devices and the external system to ensure that recorded values are consistently maintained across all components of the system. For example, the devices may be configured to transmit stored values at predetermined intervals, upon detection of network connectivity, or when placed within a docking or charging station. In an exemplary embodiment, bidirectional communication may be supported, allowing external systems to transmit status information or configuration settings back to the register devices, such as activation of strict intake and output monitoring status.

By providing integrated data transmission and connectivity capabilities, the intake and output register system allows the advantages of dedicated, point-of-care hardware documentation to be combined with the recordkeeping and accessibility benefits of centralized electronic systems. This arrangement enables accurate real-time capture at the bedside while supporting downstream storage, review, and integration with broader clinical information systems without requiring caregivers to interrupt their workflow to interact directly with separate charting interfaces.

According to an exemplary embodiment, the intake and output register system may be configured to operate in conjunction with an electronic medical record (EMR) system in a manner that supports existing clinical documentation practices while preserving the advantages of real-time, point-of-care data capture. The register devices may be configured to transmit recorded intake and output values to the EMR system in a preliminary or pending state rather than automatically entering such values as final chart entries. This arrangement allows caregivers to review and verify the transmitted information within the EMR workflow prior to committing the data to the permanent patient record.

When a caregiver records an intake or output event using one of the register devices, the corresponding data may be stored locally within the device and subsequently transmitted to the EMR system through the communication module described herein. Upon receipt, the EMR system may designate the transmitted values as pending entries associated with the relevant patient. Caregivers may then access these pending entries through the EMR interface, confirm their accuracy, and approve them for final inclusion in the patient’s intake and output chart. This process reduces the need for manual transcription while maintaining clinician oversight and compliance with established documentation protocols.

According to an exemplary embodiment, the EMR integration workflow may include periodic review cycles in which caregivers reconcile recorded values from the register devices with other clinical information. For example, at designated intervals during a nursing shift, caregivers may review pending intake and output entries within the EMR system and confirm their accuracy before finalizing documentation. This periodic reconciliation process helps ensure consistency between real-time captured data and formal chart records, while also providing an opportunity to identify and correct any discrepancies.

According to another exemplary embodiment, the EMR system may also be configured to transmit status information back to the register devices as part of a bidirectional communication arrangement. For example, when a patient is placed on strict intake and output monitoring status within the EMR system, a corresponding signal may be transmitted to the register devices to activate the input and output indicator described above. Conversely, when the strict monitoring status is discontinued, the EMR system may transmit a signal to deactivate the indicator. In this manner, the register devices remain synchronized with the patient’s documentation status within the EMR environment.

By supporting a workflow in which intake and output data are captured at the point of care and subsequently verified within the EMR system, the integration described herein combines the reliability of dedicated hardware documentation with the recordkeeping and oversight capabilities of electronic charting systems. This arrangement reduces reliance on delayed data entry, minimizes transcription errors, and aligns the register system with existing clinical documentation practices without requiring caregivers to interrupt patient care activities to interact directly with EMR interfaces at the time of each intake or output event.

In an exemplary embodiment, the intake and output register system may be configured to support continuity of care by enabling use of the register devices beyond the inpatient clinical environment, including during post-discharge monitoring in outpatient or home-care settings. In such implementations, the register devices may accompany a patient after discharge from a hospital or other care facility so that intake and output documentation may continue without interruption. This arrangement allows fluid monitoring protocols established during inpatient treatment to be maintained consistently during subsequent stages of recovery or long-term management.

In an exemplary embodiment, the register device may operate in substantially the same manner as described above for inpatient use, allowing caregivers, patients, or family members to record intake and output events using the fixed increment input controls and displays provided on the device. The same features that facilitate reliable documentation in clinical settings, such as tactile input buttons, dual displays, and correction safeguards, may thereby be leveraged in home or outpatient environments where formal charting systems may not be readily available.

The register device according to an exemplary embodiment may further be configured to transmit recorded intake and output data to a remote computing environment accessible to healthcare providers. For example, devices may communicate with a cloud-based system that stores patient-specific intake and output records and allows authorized clinicians to review such data remotely. This capability enables physicians, nurses, or care coordinators to monitor a patient’s fluid status after discharge without requiring frequent in-person visits, thereby supporting early detection of potential complications such as dehydration, fluid overload, or impaired renal function.

In an exemplary embodiment, the continuity-of-care system may include a mobile or web-based application configured to interface with the remote data storage system. Such an application may permit patients or caregivers to view recorded intake and output information, receive reminders regarding documentation, and share data with healthcare providers. The application may also provide visualization tools that present intake and output trends over time, thereby assisting providers in evaluating patient progress and making informed treatment decisions.

The continuity-of-care embodiments described herein allow the intake and output register system to function as a unified documentation platform that spans multiple stages of patient care, from inpatient hospitalization through outpatient recovery. By enabling consistent, real-time recording of fluid intake and output across care settings and facilitating remote provider access to such data, the system enhances clinical oversight, improves documentation continuity, and reduces the likelihood of information loss during transitions between care environments.

While the intake and output register system according to exemplary embodiments has been described with reference to specific structural features and operational workflows, it will be appreciated that various modifications and alternative configurations may be implemented without departing from the scope of the invention. The particular arrangements of input controls, displays, indicators, and communication features described herein are illustrative, and numerous variations are contemplated to accommodate different clinical environments, patient populations, and operational requirements.

In exemplary embodiments, the input control configuration may vary from the fixed increment button arrangement described above. For example, alternative devices may include different increment values, alternative button layouts, or configurable input settings tailored to specific clinical applications. In some implementations, the input interface may include additional tactile, visual, or audible feedback mechanisms to confirm entry of intake or output events. The relative placement, size, and labeling of input controls may also be modified to optimize usability for different caregiver workflows or patient needs.

Similarly, exemplary embodiments may employ different types of indicator systems in place of, or in addition to, the strict I&O indicator light described herein. For example, visual indicators of different colors, shapes, or illumination patterns may be used to convey various documentation statuses. In an exemplary embodiment, auditory or haptic alerts may supplement visual indicators to provide additional notification of strict monitoring conditions. The indicator system may also be configured to communicate other status information, such as device connectivity, pending data transmission, or required reconciliation actions.

The physical mounting configuration of the register devices may likewise be adapted to different care environments. While magnetic mounting and docking arrangements have been described, exemplary embodiments may include adhesive mounts, mechanical fasteners, wall-mounted brackets, or portable configurations designed for handheld use. In an exemplary embodiment, the device may be integrated into existing medical equipment, such as bed rails, bedside consoles, or mobile workstation units.

Additional exemplary embodiments may incorporate expanded power management and charging features. For example, the devices may include rechargeable battery systems with docking stations configured to provide both charging and data synchronization capabilities. In other exemplary embodiments, the devices may be powered through wired connections, removable battery modules, or alternative energy sources suitable for long-term deployment in clinical or home settings.

The communication and data transmission features described herein may also be implemented using a variety of alternative technologies. For example, the devices may utilize different wireless communication protocols, may support direct device-to-device communication, or may operate in offline modes in which data is stored locally and transmitted when connectivity becomes available. In exemplary embodiments, the system may include additional security and authentication features to protect patient data during storage and transmission.

Furthermore, while the system has been described primarily in the context of fluid intake and output documentation, the underlying register architecture may be adapted for tracking other patient-related events or clinical parameters requiring real-time, point-of-care documentation. For example, the devices may be configured to record additional patient care activities, medication administration events, or other measurable clinical inputs and outputs, provided that such adaptations remain consistent with the principles of dedicated, immediate documentation embodied in the invention.

These exemplary embodiments are contemplated as falling within the scope of the invention, and it should be understood that the specific configurations described herein are provided for illustrative purposes rather than as limitations.

According to an exemplary embodiment, the intake and output register system may be configured to facilitate real-time documentation of patient fluid intake and output events at the point of care. In a typical clinical setting, separate intake and output register devices are positioned in locations where intake and output events are most likely to occur, such as at the patient’s bedside and in a nearby bathroom. Caregivers record intake events, such as oral or intravenous fluid administration, and output events, such as urine or other fluid losses, by actuating the appropriate fixed increment input controls on the corresponding register device at or near the time the event occurs. The tactile input controls and immediate display feedback enable rapid documentation without requiring the caregiver to access a separate charting system.

When an intake or output event is recorded, the register device may update the cumulative total displayed on the primary display and, where applicable, update a secondary display associated with unmeasured or incontinent events. If an input error occurs, the caregiver may actuate the corresponding correction control to adjust the recorded value, subject to any timing safeguards that prevent inadvertent or accidental changes. At the end of a designated documentation period, such as a nursing shift, authorized personnel may reset the register totals after reviewing and reconciling the recorded values.

According to an exemplary embodiment, a patient may be designated for strict intake and output monitoring. In such cases, the strict input and output indicator integrated into the register device may be activated manually by a caregiver or automatically through a signal transmitted from an external system, such as an electronic medical record. Once activated, the indicator provides a persistent visual cue that heightened documentation attention is required. Caregivers are thereby prompted to record all intake and output events promptly using the register device, thereby reducing reliance on memory and minimizing delayed documentation.

In exemplary embodiments incorporating data transmission capabilities, recorded intake and output values may be communicated from the register device to an external system, such as a centralized server or an electronic medical record. In some implementations, transmitted data may be designated as pending entries within the electronic medical record, allowing caregivers to review and confirm the accuracy of the recorded values before finalizing documentation. Periodic reconciliation of pending entries may occur during routine clinical workflows, such as scheduled chart review intervals, to ensure consistency between real-time recorded data and formal patient records.

In continuity-of-care exemplary embodiments, the register device may continue to be used after a patient is discharged from an inpatient setting. The register device may accompany the patient to a home or outpatient environment, where intake and output events may be recorded in the same manner as in the clinical setting. Recorded data may be transmitted to a remote system accessible by healthcare providers, enabling ongoing monitoring of the patient’s fluid status and supporting timely clinical intervention when necessary.

Through these methods of use, the intake and output register system may permit consistent, immediate documentation of fluid intake and output events across various care environments. By providing a dedicated, point-of-care recording mechanism that integrates with existing documentation workflows, the system improves the reliability, accuracy, and timeliness of intake and output tracking while reducing the burden associated with traditional charting practices.

Although certain exemplary embodiments and implementations have been described herein, other embodiments and modifications will be apparent from this description. Accordingly, the inventive concept is not limited to such embodiments, but rather to the broader scope of the presented claims and various obvious modifications and equivalent arrangements.

I claim:

1. A system for real-time documentation of patient fluid intake and output events, comprising:
a first register device configured to record fluid intake events at a point of patient care; and
a second register device configured to record fluid output events at a point of patient care,
wherein each of the first and second register devices comprises one or more input controls configured to record a fluid event by incrementing a cumulative value by a predetermined unit amount, a display configured to present a cumulative total of recorded fluid events, and a correction control configured to adjust the cumulative total to account for an erroneously recorded fluid event, and
wherein the first and second register devices are configured to allow recording of fluid intake and output events substantially contemporaneously with occurrence of the events without requiring entry into an electronic medical record system.

2. The system of claim 1, wherein each register device includes separate displays for cumulative totals and unmeasured events.

3. The system of claim 1, wherein the input controls include multiple buttons corresponding to predetermined unit volumes.

4. The system of claim 1, wherein the correction control includes paired increment and decrement safeguards.

5. The system of claim 1, wherein the first register device is configured for bedside placement and the second register device is configured for placement proximate to a restroom or drainage location.

6. The system of claim 1, further comprising a strict intake and output indicator integrated into at least one of the register device housings.

7. The system of claim 6, wherein the indicator is configured for both manual and remote activation.

8. The system of claim 1, wherein at least one register device includes a mounting mechanism selected from magnetic attachment, docking engagement, or fixed mounting hardware.

9. The system of claim 1, wherein the register devices are configured to permit recording without initiation of an electronic charting session.

10. An intake and output register system for tracking a patient’s fluid balance, the intake and output register system comprising:
a first register configured to record fluid intake, the first register comprising:
a first display screen for displaying total intake and times of incontinence;
a plurality of first input buttons corresponding to numerical values for recording intake amounts and incontinence events;
a first clear/reset button for resetting the recorded intake to zero;
a plurality of first correction buttons for modifying erroneously entered data; and
a first lock-out button to prevent unintended input when activated; and
a second register configured to record fluid output, the second register comprising:
a second display screen for displaying total output and times of incontinence;
a plurality of second output buttons corresponding to numerical values for recording output amounts and incontinence events;
a second clear/reset button for resetting the recorded output to zero;
a plurality of second correction buttons for modifying erroneously entered data; and
a second lock-out button to prevent unintended input when activated.

11. The system of claim 10, wherein the intake input buttons include at least three distinct volume increments.

12. The system of claim 10, wherein the lockout control includes a time-based delay mechanism.

13. The system of claim 10, wherein the intake and output devices include independent reset functions.

14. The system of claim 10, wherein each register device is operable independently of the other.

15. The system of claim 10, wherein the input buttons include tactile or ergonomic surface features.

16. A patient fluid documentation system, comprising:
a first register device configured to record fluid intake events at a point of patient care;
a second register device configured to record fluid output events at a point of patient care; and
a communication interface integrated into at least one of the first register device or the second register device and configured to transmit recorded intake and output data from the respective register device to a remote computing device,
wherein each of the first and second register devices comprises a housing, one or more input controls configured to increment a cumulative value by a predetermined unit amount, a display configured to present the cumulative value, and a correction control configured to adjust the cumulative value.

17. The system of claim 16, wherein the communication module includes wireless communication capability.

18. The system of claim 16, wherein recorded data is stored locally prior to transmission.
